(19) United States
(12) Patent Application Publication
(10) Pub. No.: US 2022/0415524 A1
(43) Pub. Date: Dec. 29, 2022

(54) MACHINE LEARNING-BASED ADJUSTMENT OF EPIDEMIOLOGICAL MODEL PROJECTIONS WITH FLEXIBLE PREDICTION HORIZON

(71) Applicant: International Business Machines Corporation, Armonk, NY (US)

(72) Inventors: Sarah Kefayati, San Francisco, CA (US); PRITHWISH CHAKRABORTY, New York, NY (US); Ajay Ashok Deshpande, Pleasantville, NY (US); Vishrawas Gopalakrishnan, Cambridge, MA (US); Jianying Hu, Bronx, NY (US); Hu Trombley Huang, Maple Grove, MN (US); Gretchen Jackson, Nashville, TN (US); Xuan Liu, Yorktown Heights, NY (US); SAYALI NAVALEKAR, Westford, MA (US); Raman Srinivasan, Plano, TX (US)

(21) Appl. No.: 17/361,717

(22) Filed: Jun. 29, 2021

(51) Int. Cl. G16H 50/80; G06N 20/00; G16H 50/70; G16H 50/20; G16H 40/20

(52) U.S. Cl. CPC G16H 50/80; G06N 20/00; G16H 50/70; G16H 50/20; G16H 40/20; G16H 10/40

(57) ABSTRACT

In an approach for building a machine learning model with a flexible prediction horizon, a processor gathers statistical data related to a disease from one or more regional sources. A processor clusters the statistical data according to a plurality of localized regional source similarity criteria and a plurality of region criteria. A processor builds a plurality of training models based on the clustered statistical data. A processor builds a plurality of feature vectors based on the plurality of localized regional source similarity criteria and the plurality of region criteria. A processor trains the plurality of training models separately against the plurality of feature vectors. A processor selects a best performing training model for each of the plurality of localized regional source similarity criteria and the plurality of region criteria based on a performance criterion. A processor tests the best performing training model to predict one or more future outcomes.

### BACKGROUND OF THE INVENTION

The present invention relates generally to the field of data processing, and more particularly to a machine learning-based adjustment of epidemiological model projections with a flexible prediction horizon.

During a pandemic, such as the COVID-19 pandemic, epidemiologists work to fully understand the nature of the pandemic and to track and to slow its spread. Epidemiologists have a myriad of potential sources of data available to them for this purpose. Namely, epidemiologists have access to notifiable disease case data through mandatory reporting by providers and laboratories. Existing data on health risk behaviors is available to them from population-based surveys, however, these surveys are generally conducted among a small proportion of the total population and are de-identified. Additionally, epidemiologists have access to other existing sources of data, such as death certificates, cover disease outcomes.

In addition to collecting data, epidemiologists conduct public health surveillance to track the spread of a disease causing the pandemic. Public health surveillance is the systematic collection, analysis, and interpretation of health data. Public health surveillance allows epidemiologists to calculate incidence (i.e., the number of new cases reported over a specific period of time), prevalence (i.e., the number of cases at one specific point in time), hospitalizations (i.e., the number of cases resulting in hospitalizations), and deaths (i.e., the number of cases resulting in deaths). However, public health surveillance isn't just about counting cases. Patient specific information can be collected to learn more about the course and spread of a disease. For example, data collected from medical records (i.e., chart abstractions) can tell the epidemiologists more about COVID-19 patients and the course of a patient's disease. The data collected from medical records might include socio-demographic information (i.e., age), as well as symptoms, treatments, and health outcomes.

A key role of epidemiologists during a pandemic is to estimate the burden of disease. The burden of disease is the impact or other health outcomes of the disease on a population. This includes the number of infections, illnesses, medical visits, hospitalizations, and deaths. The estimated burden of disease on a population is often presented in an epidemiological model. One of the commonly used epidemiological models is the susceptible-exposed-infectious-removed model.

Current epidemiological models produced have shown potential in providing an impact on the different mitigation scenarios related to a pandemic. However, one of the major drawbacks of the epidemiological models is the overestimation of the number of future cases. The overestimation of the number of future cases in these epidemiological models can led to public uncertainty and most importantly the misuse of resources by the government and by health care providers.

### SUMMARY

Aspects of an embodiment of the present invention disclose a method, computer program product, and computer system for building a machine learning model to adjust epidemiological model projections with a flexible prediction horizon. A processor gathers statistical data related to a disease from one or more regional sources. A processor clusters the statistical data related to the disease according to a plurality of localized regional source similarity criteria and a plurality of region criteria. A processor builds a plurality of training models based on the clustered statistical data. A processor trains the plurality of training models with a time variant feature. A processor builds a plurality of feature vectors based on the plurality of localized regional source similarity criteria and the plurality of region criteria. A processor trains the plurality of training models separately against the plurality of feature vectors in order to predict the one or more outcomes for each of the plurality of localized regional source similarity criteria and the plurality of region criteria. A processor selects a best performing training model for each of the plurality of localized regional source similarity criteria and the plurality of region criteria based on a performance criterion. A processor selects test data for each of the plurality of localized regional source similarity criteria and the plurality of region criteria based on the best performing training model from historical data reported on a desired outcome. A processor updates the best performing training model. A processor tests the best performing training model with the test data selected to predict the one or more future outcomes.

In some aspects of an embodiment of the present invention, the statistical data includes at least one of a number of confirmed cases of the disease, a number of confirmed fatalities from the disease, one or more test results, a set of data regarding one or more symptoms of the disease, government policy responses to the disease and social interventions, and a previous history of disease in one or more regions of a country.

In some aspects of an embodiment of the present invention, the localized regional source similarity criteria includes phase matching based on a daily case time series curve; similarities in socio-demographic features; comorbidities; mobility; and government policy responses to the disease and social interventions.

In some aspects of an embodiment of the present invention, the region criteria by which the statistical data is clustered includes state boundaries and national boundaries.

In some aspects of an embodiment of the present invention, a processor inputs one or more Susceptible-Exposed-Infectious-Removed projections, one or more autoregressive features, one or more sociodemographic features, and one or more time-dependent features into the plurality of feature vectors.

In some aspects of an embodiment of the present invention, the one or more outcomes is one or more of a daily number of confirmed cases of the disease, a daily number of hospitalizations because of the disease, a daily number of intensive care unit beds occupied by one or more patients with the disease, and a daily number of deaths as a result of the disease.

In some aspects of an embodiment of the present invention, a processor conducts a walk-forward expanding window method of a train test split.

In some aspects of an embodiment of the present invention, a processor uses a grid search in order to find a best score. A processor uses mean absolute error as a training scorer. A processor predicts a final score using R².

In some aspects of an embodiment of the present invention, the performance criterion is either a mean absolute percentage error or a root mean square.

In some aspects of an embodiment of the present invention, subsequent to testing the best performing training model with the test data selected to predict the one or more future outcomes, a processor outputs the one or more future outcomes to a user as an alert notification.

### BRIEF DESCRIPTION OF THE DRAWINGS

FIG. 1 is a functional block diagram illustrating a distributed data processing environment, in accordance with an embodiment of the present invention;

FIG. 2 is a flowchart illustrating the operational steps for a setup component of an epidemiological model projection adjustment program, on a server within the distributed data processing environment of FIG. 1, in accordance with an embodiment of the present invention;

FIG. 3 is a flowchart illustrating the operational steps of the epidemiological model projection adjustment program, on the server within the distributed data processing environment of FIG. 1, in accordance with an embodiment of the present invention; and

FIG. 4 is a block diagram illustrating the components of a computing device in the distributed data processing environment of FIG. 1, in accordance with an embodiment of the present invention.

### DETAILED DESCRIPTION

Epidemiologists typically present their findings in epidemiological models. One of the commonly used epidemiological models is the susceptible-exposed-infectious-removed compartmental model. The epidemiological models can be used to help with different mitigation scenarios for a pandemic. However, embodiments of the present invention recognize that one of the major drawbacks of epidemiological models is the overestimation of the number of future cases of a disease in a pandemic. The overestimation of the number of future cases can led to public uncertainty and most importantly, in this case, the misuse of resources by the government and by health care providers. Therefore, embodiments of the present invention recognize that it is imperative to more accurately forecast the number of future cases and, in turn, the health care system capacity requirements so that the government and the health care providers can properly respond to future pandemics.

Embodiments of the present invention provide a system and method to build an artificial intelligence model (i.e., a machine learning model) with a flexible prediction horizon (hereinafter referred to as a time frame). The susceptible-exposed-infectious-removed compartmental model forms the basis of the artificial intelligence model that is built. Embodiments of the present invention provide a system and method to predict the spread of a disease in one or more counties in one or more regions of a country using the artificial intelligence model.

Implementation of embodiments of the present invention may take a variety of forms, and exemplary implementation details are discussed subsequently with reference to the Figures.

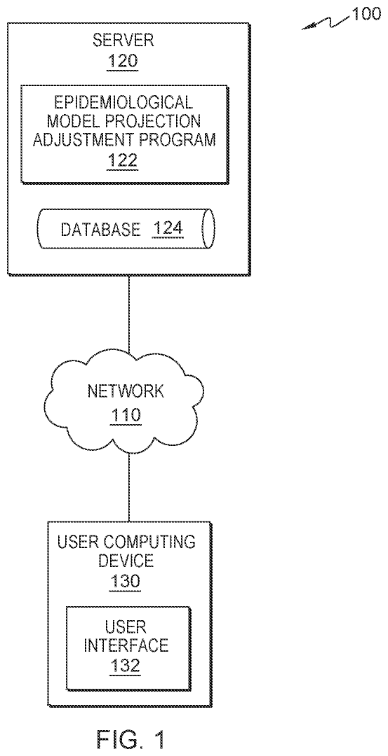

FIG. 1 is a functional block diagram illustrating a distributed data processing environment, generally designated 100, in accordance with an embodiment of the present invention. In the depicted embodiment, distributed data processing environment 100 includes server 120 and user computing device 130, interconnected over network 110. Distributed data processing environment 100 may include additional servers, computers, computing devices, IoT sensors, and other devices not shown. The term “distributed” as used herein describes a computer system that includes multiple, physically distinct devices that operate together as a single computer system. FIG. 1 provides only an illustration of one embodiment of the present invention and does not imply any limitations with regard to the environments in which different embodiments may be implemented. Many modifications to the depicted environment may be made by those skilled in the art without departing from the scope of the invention as recited by the claims.

Network 110 operates as a computing network that can be, for example, a telecommunications network, a local area network (LAN), a wide area network (WAN), such as the Internet, or a combination of the three, and can include wired, wireless, or fiber optic connections. Network 110 can include one or more wired and/or wireless networks capable of receiving and transmitting data, voice, and/or video signals, including multimedia signals that include voice, data, and video information. In general, network 110 can be any combination of connections and protocols that will support communications between server 120, user computing device 130, and other computing devices (not shown) within distributed data processing environment 100.

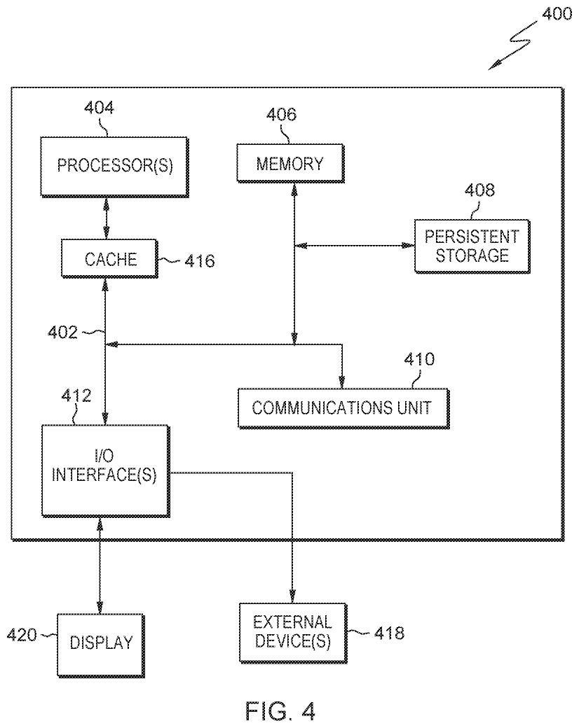

Server 120 operates to run epidemiological model projection adjustment program 122 and to send and/or store data in database 124. In an embodiment, server 120 can send data from database 124 to user computing device 130. In an embodiment, server 120 can receive data in database 124 from user computing device 130. In one or more embodiments, server 120 can be a standalone computing device, a management server, a web server, a mobile computing device, or any other electronic device or computing system capable of receiving, sending, and processing data and capable of communicating with user computing device 130 via network 110. In one or more embodiments, server 120 can be a computing system utilizing clustered computers and components (e.g., database server computers, application server computers, etc.) that act as a single pool of seamless resources when accessed within distributed data processing environment 100, such as in a cloud computing environment. In one or more embodiments, server 120 can be a laptop computer, a tablet computer, a netbook computer, a personal computer, a desktop computer, a personal digital assistant, a smart phone, or any programmable electronic device capable of communicating with user computing device 130 and other computing devices (not shown) within distributed data processing environment 100 via network 110. Server 120 may include internal and external hardware components, as depicted and described in further detail in FIG. 4.

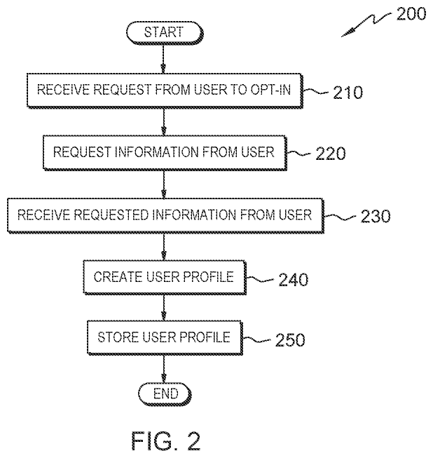

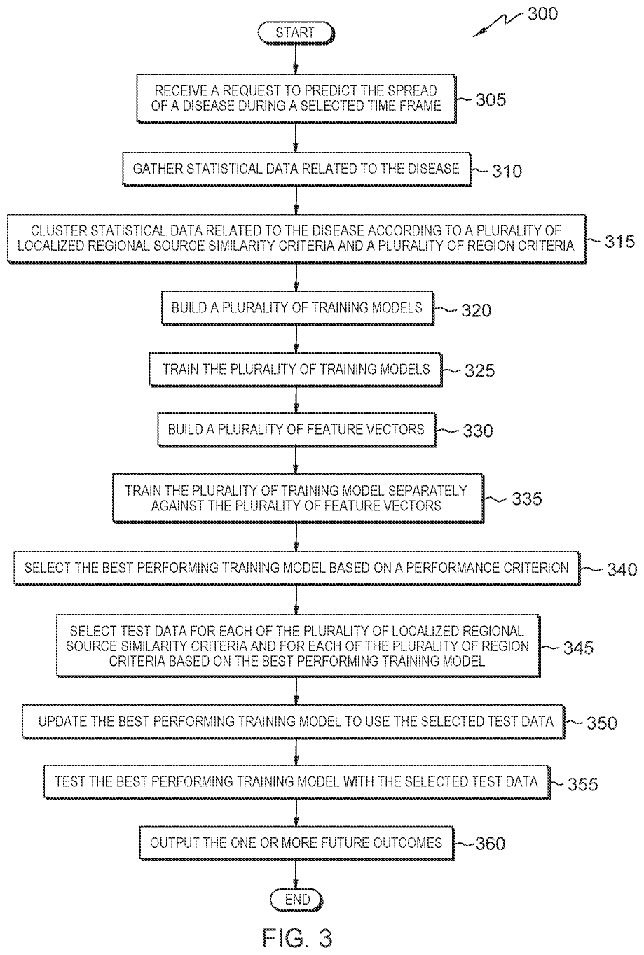

Epidemiological model projection adjustment program 122 operates to build an artificial intelligence model (i.e., a machine learning model) with an adjustable prediction time frame. The susceptible-exposed-infectious-removed compartmental model forms the basis of the artificial intelligence model that is built. Epidemiological model projection adjustment program 122 operates to predict the spread of a disease in one or more counties in one or more regions of a country during the selected prediction time frame using the artificial intelligence model. Counties can include regions or any smaller unit of a country. In the depicted embodiment, epidemiological model projection adjustment program 122 is a standalone program. In another embodiment, epidemiological model projection adjustment program 122 may be integrated into another software product. In the depicted embodiment, epidemiological model projection adjustment program 122 resides on server 120. In another embodiment, epidemiological model projection adjustment program 122 may reside on user computing device 130 or on another computing device (not shown), provided that epidemiological model projection adjustment program 122 has access to network 110. In an embodiment, a user opts-in to epidemiological model projection adjustment program 122 and sets up a user profile with epidemiological model projection adjustment program 122. The operational steps of the setup component of epidemiological model projection adjustment program 122 are depicted and described in further detail with respect to FIG. 2. The operational steps of epidemiological model projection adjustment program 122 are depicted and described in further detail with respect to FIG. 3.

Database 124 operates as a repository for data received, used, and/or generated by epidemiological model projection adjustment program 122. A database is an organized collection of data. Data includes, but is not limited to, information about user preferences, alert notification preferences, feature preferences, time frame preferences, confidence interval preferences, and region-specific information; information necessary to create a user profile; the user profile; statistical data related to the disease from one or more regional sources; a plurality of training models; a plurality of feature vectors based on a plurality of localized regional source similarity criteria and a plurality of region criteria; a training model selected as the best performing and related accuracy criteria; test data for each of the localized regional sources; one or more future outcomes; and any other data received, used, and/or generated by epidemiological model projection adjustment program 122.

Database 124 can be implemented with any type of device capable of storing data and configuration files that can be accessed and utilized by server 120, such as a hard disk drive, a database server, or a flash memory. In an embodiment, database 124 is accessed by epidemiological model projection adjustment program 122 to store and/or to access the data. In the depicted embodiment, database 124 resides on server 120. In another embodiment, database 124 may reside on another computing device, server, cloud server, or spread across multiple devices elsewhere (not shown) within distributed data processing environment 100, provided that epidemiological model projection adjustment program 122 has access to database 124.

The present invention may contain various accessible data sources, such as database 124, that may include personal and/or confidential company data, content, or information the user wishes not to be processed. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal and/or confidential company data. Epidemiological model projection adjustment program 122 enables the authorized and secure processing of personal data.

Epidemiological model projection adjustment program 122 provides informed consent, with notice of the collection of personal and/or confidential company data, allowing the user to opt-in or to opt-out of processing personal and/or confidential company data. Consent can take several forms. Opt-in consent can impose on the user the opportunity to take an affirmative action before personal and/or confidential company data is processed. Alternatively, opt-out consent can impose on the user the opportunity to take an affirmative action to prevent the processing of personal and/or confidential company data before personal and/or confidential company data is processed. Epidemiological model projection adjustment program 122 provides information regarding personal and/or confidential company data and the nature (e.g., type, scope, purpose, duration, etc.) of the processing. Epidemiological model projection adjustment program 122 provides the user with copies of stored personal and/or confidential company data. Epidemiological model projection adjustment program 122 allows the correction or completion of incorrect or incomplete personal and/or confidential company data. Epidemiological model projection adjustment program 122 allows for the immediate deletion of personal and/or confidential company data.

In an embodiment, the user of user computing device 130 can register with server 120 (e.g., via a corresponding application). For example, the user completes a registration process, provides information, and authorizes the collection and analysis (i.e., opts-in) of relevant data on identified computing devices, (e.g., user computing device 130) by server 120 (e.g., via epidemiological model projection adjustment program 122). In various embodiments, a user can opt-in or opt-out of certain categories of data collection. For example, the user can opt-in to provide all requested information, a subset of requested information, or no information. In one example scenario, the user opts-in to provide time-based information, but opts-out of providing location-based information (on all or a subset of computing devices associated with the user). In another embodiment, the user can define which forms of data server 120 can utilize in determining a malfunction or system error. In another embodiment, the user can opt-out of the determination and/or analysis of a malfunction or system error entirely. In such embodiments, the user can interact with chatbots in the messaging session normally (i.e., without assistance/interaction with epidemiological model projection adjustment program 122). Such preferences can be stored in database 124.

User computing device 130 operates to run user interface 132 through which a user can interact with epidemiological model projection adjustment program 122 on server 120. In an embodiment, user computing device 130 is a device that performs programmable instructions. For example, user computing device 130 may be an electronic device, such as laptop computer, a tablet computer, a netbook computer, a personal computer, a desktop computer, a smart phone, or any programmable electronic device capable of running user interface 132 and of communicating (i.e., sending and receiving data) with epidemiological model projection adjustment program 122 via network 110. In general, user computing device 130 represents any programmable electronic device or a combination of programmable electronic devices capable of executing machine readable program instructions and communicating with other computing devices (not shown) within distributed data processing environment 100 via network 110. In the depicted embodiment, user computing device 130 includes an instance of user interface 132. User computing device 130 may include components as described in further detail in FIG. 4.

User interface 132 operates as a local user interface between epidemiological model projection adjustment program 122 on server 120 and a user of user computing device 130. In some embodiments, user interface 132 is a graphical user interface (GUI), a web user interface (WUI), and/or a voice user interface (VUI) that can display (i.e., visually) or present (i.e., audibly) text, documents, web browser windows, user options, application interfaces, and instructions for operations sent from epidemiological model projection adjustment program 122 to a user via network 110. User interface 132 can also display or present alerts including information (such as graphics, text, and/or sound) sent from epidemiological model projection adjustment program 122 to a user via network 110. In an embodiment, user interface 132 is capable of sending and receiving data (i.e., to and from epidemiological model projection adjustment program 122 via network 110, respectively).

FIG. 2 is a flowchart, generally designated 200, illustrating the operational steps for a setup component of epidemiological model projection adjustment program 122 in distributed data processing environment 100 of FIG. 1, in accordance with an embodiment of the present invention. In an embodiment, epidemiological model projection adjustment program 122 completes a one-time setup with a user. The one-time setup allows for epidemiological model projection adjustment program 122 to capture relevant information about the user to create a user profile. In an embodiment, epidemiological model projection adjustment program 122 receives a request from the user to opt-in. In an embodiment, epidemiological model projection adjustment program 122 requests information from the user. In an embodiment, epidemiological model projection adjustment program 122 receives the requested information from the user. In an embodiment, epidemiological model projection adjustment program 122 creates a user profile. In an embodiment, epidemiological model projection adjustment program 122 stores the user profile. It should be appreciated that the process depicted in FIG. 2 illustrates one possible iteration of epidemiological model projection adjustment program 122, which may be repeated for each opt-in request received by epidemiological model projection adjustment program 122.

In step 210, epidemiological model projection adjustment program 122 receives a request from a user to opt-in. A user may include, but is not limited to, a government agency with regards to making policy and/or developing guidelines to mitigate outbreaks and a health care provider (e.g., hospital, urgent care center, clinic, pharmacy, and laboratory) with regards to allocating resources, treating infected patients, educating the public about social distancing measures, and/or testing potential treatments and vaccines. In an embodiment, epidemiological model projection adjustment program 122 receives a request from a user to opt-in to epidemiological model projection adjustment program 122. In an embodiment, epidemiological model projection adjustment program 122 receives a request from a user to opt-in to epidemiological model projection adjustment program 122 through user interface 132 of user computing device 130. By opting-in, the user agrees to share data with database 124.

In step 220, epidemiological model projection adjustment program 122 requests information from the user. In an embodiment, epidemiological model projection adjustment program 122 requests information from the user through user interface 132 of user computing device 130. Information requested from the user includes, but is not limited to, information about user preferences (e.g., general user system settings such as alert notifications for user computing device 130); information about alert notification preferences; information necessary to create a user profile; information about feature preferences (i.e., the order of importance of the features and what information on a particular feature is most important for the user to receive); information about the start date of a time frame prediction; information about time frame preferences (e.g., 1-day, 3-days, 1-week, 2-weeks, or 3-weeks); information about confidence interval preferences (e.g., 75%, 80%, or 85%); and region-specific information including socio-demographic features (e.g., age, employment, education, income, and birth and death rates), as well as symptom, treatment, and health outcome information.

In an embodiment, epidemiological model projection adjustment program 122 enables a user to select from a list of one or more groups with which the user would like to be grouped. The one or more groups include, but are not limited to, a government agency, a healthcare provider, and a university. In an embodiment, epidemiological model projection adjustment program 122 enables a user to select from a list of one or more groups in order to cluster the user with similar users nationwide. In an embodiment, epidemiological model projection adjustment program 122 enables a user to select the one or more groups through user interface 132 of user computing device 130. In an embodiment, responsive to receiving the request from the user to opt-in, epidemiological model projection adjustment program 122 requests information from the user.

In step 230, epidemiological model projection adjustment program 122 receives the requested information from the user. In an embodiment, epidemiological model projection adjustment program 122 receives the requested information from the user through user interface 132 of user computing device 130. In one or more embodiments, epidemiological model projection adjustment program 122 provides suggestions to the user based on the information received. For example, based on the confidence interval selected by a user, epidemiological model projection adjustment program 122 provides a suggestion on the appropriate time frame to select. In an embodiment, responsive to requesting information from the user, epidemiological model projection adjustment program 122 receives the requested information from the user.

In step 240, epidemiological model projection adjustment program 122 creates a user profile. In an embodiment, epidemiological model projection adjustment program 122 creates a user profile for the user. In an embodiment, epidemiological model projection adjustment program 122 creates a user profile with information input by the user during setup regarding the user as well as user preferences, alert notification preferences, feature preferences, time frame preferences, confidence interval preferences, and region-specific information. In an embodiment, responsive to receiving the requested information from the user, epidemiological model projection adjustment program 122 creates a user profile.

In step 250, epidemiological model projection adjustment program 122 stores the user profile. In an embodiment, epidemiological model projection adjustment program 122 stores the user profile in a database, e.g., database 124. In an embodiment, responsive to creating the user profile, epidemiological model projection adjustment program 122 stores the user profile.

FIG. 3 is a flowchart, generally designated 300, illustrating the operational steps of epidemiological model projection adjustment program 122 on server 120 in distributed data processing environment 100 of FIG. 1, in accordance with an embodiment of the present invention. In an embodiment, epidemiological model projection adjustment program 122 operates to build an artificial intelligence model (i.e., a machine learning model) with an adjustable prediction time frame. The approach to building the artificial intelligence model is a model-agnostic approach. In an embodiment, epidemiological model projection adjustment program 122 operates to predict the spread of a disease in one or more counties in one or more regions of a country during the selected prediction time frame using the artificial intelligence model. It should be appreciated that the process depicted in FIG. 3 illustrates one possible iteration of epidemiological model projection adjustment program 122, which may be repeated for each request to predict the spread of a disease in one or more counties in one or more regions of a country during a selected prediction time frame received by epidemiological model projection adjustment program 122 or for each change detected that may alter the prediction of the spread of a disease.

In step 305, epidemiological model projection adjustment program 122 receives a request. In an embodiment, epidemiological model projection adjustment program 122 receives a request to predict the spread of a disease in one or more counties in one or more regions of a country during a selected time frame. In an embodiment, epidemiological model projection adjustment program 122 receives a request from a user manually inputted through user interface 132 of user computing device 130.

For example, a hospital is receiving a higher number of patients in a particular socio-demographic group (e.g., older adults living in congregate settings) than the hospital has previously received. The hospital inputs a request to predict the spread of a disease in one or more counties in one or more regions of the country during the selected time frame through user interface 132 of user computing device 130.

In another embodiment, epidemiological model projection adjustment program 122 initiates the prediction of the spread of a disease in one or more counties in one or more regions of a country during a selected time frame automatically. In an embodiment, epidemiological model projection adjustment program 122 initiates the prediction of the spread of a disease automatically on the start date of the selected time frame indicated by the user during the setup of epidemiological model projection adjustment program 122. In one or more embodiments, epidemiological model projection adjustment program 122 initiates the prediction of the spread of a disease automatically when epidemiological model projection adjustment program 122 detects a change that may alter the prediction of the spread of a disease.

For example, if epidemiological model projection adjustment program 122 detects a change in the pace of the distribution of the vaccines for the disease, then epidemiological model projection adjustment program 122 initiates the prediction of the spread of a disease automatically. In another example, if epidemiological model projection adjustment program 122 detects a change in the number of students returning to the classroom for in-person instruction, then epidemiological model projection adjustment program 122 initiates the prediction of the spread of a disease automatically.

In step 310, epidemiological model projection adjustment program 122 gathers statistical data. In an embodiment, epidemiological model projection adjustment program 122 gathers statistical data related to the disease. Statistical data includes, but is not limited to, a number of confirmed cases of the disease (i.e., daily, weekly, biweekly, and/or cumulative number of confirmed cases), a number of confirmed fatalities from the disease (i.e., daily, weekly, biweekly, and/or cumulative number of confirmed fatalities), one or more test results (i.e., the type of test, the positive rate, the scale of testing, the total number of tests performed per confirmed case, the total number of tests performed relative to the size of the population), data regarding the symptoms of the disease (e.g., typical symptoms, average duration of symptoms, standard deviation of the average duration of symptoms, severity of symptoms, average age of the infected population displaying symptoms), government policy responses to the disease and social interventions (i.e., school closures, workplace closures, cancellation of public events, restrictions on public gatherings, stay-at-home requirements, closures of public transportation, restrictions on international and domestic travel, face covering requirements, testing and contact tracing, vaccination policy, public information campaigns, and income support and debt relief), and previous history of the disease in one or more regions of the country. In an embodiment, epidemiological model projection adjustment program 122 gathers statistical data related to the disease from one or more regional sources, e.g., Spatiotemporal Epidemiological Modeler, National Vital Statistics System, prior ground truth reports (e.g., CDC daily reports), and hereditary database market scans. In an embodiment, epidemiological model projection adjustment program 122 stores the statistical data related to the disease from the one or more regional sources in a database, e.g., database 124. In an embodiment, responsive to receiving a request, epidemiological model projection adjustment program 122 gathers statistical data.

In step 315, epidemiological model projection adjustment program 122 clusters the statistical data related to the disease. In an embodiment, epidemiological model projection adjustment program 122 clusters the statistical data related to the disease according to a plurality of localized regional source similarity criteria and a plurality of region criteria (i.e., according to static and dynamic criteria). The localized regional source similarity criteria includes, but is not limited to, phase matching based on a daily case time series curve, socio-demographic features (e.g., age, employment, education, income, and birth and death rates), comorbidities (i.e., the simultaneous presence of two or more diseases or medical conditions in a patient), mobility (i.e., the level of commute activities traced in the region-based topology), and government policy responses to the disease and social interventions. The region criteria by which the statistical data is clustered includes, but is not limited to, state boundaries (i.e., inclusive of all counties within the boundary of a state) and national boundaries (i.e., inclusive of all counties in the one or more regions of the country). In an embodiment, responsive to gathering statistical data, epidemiological model projection adjustment program 122 clusters the statistical data related to the disease.

For example, state A experienced its first wave of infections of a disease during a pandemic in March and April 2020. State B, on the other hand, did not experience its first wave of infections of the same disease until June and July 2020. Even though state A and state B hit their first wave of infections during two different time frames, epidemiological model projection adjustment program 122 recognized the similarity that existed between state A and state B. Because of the similarity that existed, epidemiological model projection adjustment program 122 used the data from state A to cluster the statistical data related to the disease from one or more counties in state B by phase matching. In another example, epidemiological model projection adjustment program 122 clusters the statistical data related to the disease in all of the counties in New York together as one state cluster and the statistical data related to the disease in all of the counties in Florida together as one state cluster. In yet another example, epidemiological model projection adjustment program 122 clusters the statistical data related to the disease in all of counties in all of fifty states of the United States, including New York and Florida, together as one national cluster.

In step 320, epidemiological model projection adjustment program 122 builds a plurality of training models. In an embodiment, epidemiological model projection adjustment program 122 builds a plurality of training models based on the clustering strategy used in step 315, i.e., clustering the statistical data related to the disease according to the plurality of localized regional source similarity criteria and the plurality of region criteria, i.e., clustering the statistical data related to the disease by phase matching based on a daily case time series curve, by socio-demographic features, by comorbidities, by mobility, by government policy responses to the disease and social interventions, by state boundaries, and by national boundaries. In an embodiment, epidemiological model projection adjustment program 122 stores the plurality of training models in a database, e.g., database 124. In an embodiment, responsive to clustering the statistical data related to the disease, epidemiological model projection adjustment program 122 builds a plurality of training models.

In step 325, epidemiological model projection adjustment program 122 trains the plurality of training models. In an embodiment, epidemiological model projection adjustment program 122 trains the plurality of training models with a time variant feature. The time variant feature supports the time frame selected. The time frame may be, but is not limited to, 1-day, 3-days, 1-week, 2-weeks, or 3-weeks. The time frame is equal to the number of days or weeks selected as the time frame. In an embodiment, epidemiological model projection adjustment program 122 trains the plurality of training models in order to predict one or more outcomes. Outcomes may be, but are not limited to, a daily number of confirmed cases of the disease, a daily number of hospitalizations because of the disease, a daily number of intensive care unit (ICU) beds occupied by patients with the disease, and a daily number of deaths as a result of the disease. In an embodiment, responsive to building a plurality of training models, epidemiological model projection adjustment program 122 trains the plurality of training models.

In step 330, epidemiological model projection adjustment program 122 builds a plurality of feature vectors. In an embodiment, epidemiological model projection adjustment program 122 builds the plurality of feature vectors based on the time frame selected. In an embodiment, epidemiological model projection adjustment program 122 inputs Susceptible-Exposed-Infectious-Removed (SEIR) projections into the plurality of feature vectors from an on-premise model developed by researchers. In one or more embodiments, epidemiological model projection adjustment program 122 inputs Susceptible-Exposed-Infectious-Removed (SEIR) projections into the plurality of feature vectors from an open-source model. In an embodiment, epidemiological model projection adjustment program 122 inputs autoregressive features into the plurality of feature vectors. Autoregressive features are the history of a disease over a period of time. For example, epidemiological model projection adjustment program 122 is predicting the number of cases of the disease for the following day. The autoregressive features are the number of cases accounted for the previous day and the previous two days. In an embodiment, epidemiological model projection adjustment program 122 inputs sociodemographic features and time-dependent features into the plurality of feature vectors.

In an embodiment, epidemiological model projection adjustment program 122 builds the plurality of feature vectors based on the plurality of localized regional source similarity criteria and the plurality of region criteria. In an embodiment, epidemiological model projection adjustment program 122 stores the plurality of feature vectors in a database, e.g., database 124. In an embodiment, responsive to training the plurality of training models, epidemiological model projection adjustment program 122 builds a plurality of feature vectors.

In step 335, epidemiological model projection adjustment program 122 further trains the plurality of training models separately against the plurality of feature vectors. In an embodiment, epidemiological model projection adjustment program 122 further trains the plurality of training models separately against the plurality of feature vectors in order to predict the one or more outcomes for each of the plurality of localized regional source similarity criteria and for each of the plurality of region criteria. In an embodiment, epidemiological model projection adjustment program 122 further trains the plurality of training models separately against the plurality of feature vectors in order to produce a plurality of models, e.g., four models, i.e., a single model for each clustering strategy.

In an embodiment, epidemiological model projection adjustment program 122 conducts a train-test split. In an embodiment, epidemiological model projection adjustment program 122 conducts a train-test split with the plurality of models produced. In an embodiment, epidemiological model projection adjustment program 122 conducts a train-test split that is date-dependent. The train-test split is a technique for evaluating the performance of a machine learning algorithm. The train-test split procedure involves taking a dataset and dividing it into two subsets. The first subset is used to fit the model and is referred to as the training dataset. The second subset is not used to train the model; instead, the input element of the dataset is provided to the model, then predictions are made and compared to the expected values. The second dataset is used to evaluate the fit of the machine learning model and is referred to as the test dataset. The objective is to estimate the performance of the machine learning model on new data, i.e., data that is not used to train the model. In practice, the machine learning model is fit on available data with known inputs and outputs, then predictions are made on new examples in the future where there are no expected outputs or target values.

The walk-forward is a method of the train-test split. The walk-forward method evaluates the performance of a machine learning algorithm during a given time frame. The time series includes a cutoff date. The walk-forward method trains the data that comes before the cutoff date and tests the data that comes after the cutoff date. The training period is much longer than the testing period, e.g., the training period takes up 80% of the time and the testing period takes up 20% of the time. The walk-forward method can be performed with either a rolling window or an expanding window. A rolling window means that there is a specified window of time for training, i.e., as the cutoff date moves forward, the historic data falls out of training. An expanding window means that all of the historic data from the specified start date to the specified end date is included in training.

In an embodiment, epidemiological model projection adjustment program 122 conducts a walk-forward expanding window method of the train test split. In one or more embodiments, epidemiological model projection adjustment program 122 conducts a walk-forward rolling window method of the train test split.

In an embodiment, epidemiological model projection adjustment program 122 uses a grid-search in order to find the best scoring model of the train-test split. The training scorer is mean absolute error (MAE) and R² is used to predict the final score.

In an embodiment, responsive to building a plurality of feature vectors, epidemiological model projection adjustment program 122 trains the plurality of training models separately against the plurality of feature vectors.

For example, epidemiological model projection adjustment program 122 trains five training models (i.e., M1, M2, M3, M4, and M5) against the plurality of feature vectors using a selected time frame. Epidemiological model projection adjustment program 122 further trains the five models using train-test split with a walk-forward expanding window regimen and a walk-forward rolling window regimen. Epidemiological model projection adjustment program 122 trains the five training models by training for one day and testing for one day, training for two days and testing for one day, training for three days of training and testing for one day, training for four days and testing for one day, and so on. Epidemiological model projection adjustment program 122 selects the best performing model and then implements the best performing model. The model selected includes all of the prior tested days as well as projections on the days that epidemiological model projection adjustment program 122 has not yet tested (e.g., the next day, the next three days, or the next week). More specifically, for M1, epidemiological model projection adjustment program 122 set t equal to one (i.e., one day). For M2, epidemiological model projection adjustment program 122 set t equal to two (i.e., two days). Epidemiological model projection adjustment program 122 expanded the testing window to include M1 and M2, the first day and the second day, and used the third day as the test.

In step 340, epidemiological model projection adjustment program 122 selects the best performing training model. In an embodiment, epidemiological model projection adjustment program 122 selects the best performing training model for each of the plurality of localized regional sources similarity criteria and for each of the plurality of region criteria. In an embodiment, epidemiological model projection adjustment program 122 selects the best performing training model based on a performance criterion. The performance criterion can either be the mean absolute percentage error (MAPE) or the root mean square (RMS). In an embodiment, epidemiological model projection adjustment program 122 stores the training model selected as the best performing and the performance criterion in a database, e.g., database 124. In an embodiment, responsive to training the plurality of training models separately against the plurality of feature vectors, epidemiological model projection adjustment program 122 selects the best performing training model.

In step 345, epidemiological model projection adjustment program 122 selects test data. In an embodiment, epidemiological model projection adjustment program 122 selects test data for each of the plurality of localized regional source similarity criteria and for each of the plurality of region criteria. In an embodiment, epidemiological model projection adjustment program 122 selects test data for each of the localized regional sources based on the best performing training model. In an embodiment, epidemiological model projection adjustment program 122 selects test data from the historical data reported on a desired outcome. For example, epidemiological model projection adjustment program 122 is predicting the daily number of hospitalizations due to a disease for the upcoming week. The previously reported data on the daily number of hospitalizations due to the disease is used. In an embodiment, epidemiological model projection adjustment program 122 stores the test data for each of the plurality of localized regional source similarity criteria and for each of the plurality of region criteria in a database, e.g., database 124. In an embodiment, responsive to selecting the best performing training model, epidemiological model projection adjustment program 122 selects test data.

In step 350, epidemiological model projection adjustment program 122 updates the best performing training model. In an embodiment, epidemiological model projection adjustment program 122 updates the best performing training model to use the test data selected in step 345. In an embodiment, responsive to selecting test data, epidemiological model projection adjustment program 122 updates the best performing training model.

In step 355, epidemiological model projection adjustment program 122 tests the best performing training model with the selected test data. In an embodiment, epidemiological model projection adjustment program 122 tests the best performing training model with the selected test data in order to predict the one or more future outcomes (i.e., the daily number of confirmed cases of the disease, the daily number of hospitalizations because of the disease, the daily number of ICU beds occupied by patients with the disease, and the daily number of deaths as a result of the disease).

In an embodiment, epidemiological model projection adjustment program 122 converts the one or more future outcomes based on the population of one or more counties in one or more regions of a country and based on the daily number of cases. For example, epidemiological model projection adjustment program 122 first predicts the daily number of hospitalizations. Epidemiological model projection adjustment program 122 then predicts the daily number of occupied ICU beds through a conversion based on the historical data of hospitalizations to ICU beds for the one or more regions of the country. Epidemiological model projection adjustment program 122 uses this number to complete the conversion.

In an embodiment, epidemiological model projection adjustment program 122 stores the one or more future outcomes in a database, e.g., database 124. In an embodiment, responsive to updating the best performing training model, epidemiological model projection adjustment program 122 tests the best performing training model with the selected test data.

In step 360, epidemiological model projection adjustment program 122 outputs the one or more future outcomes. In an embodiment, epidemiological model projection adjustment program 122 outputs the one or more future outcomes predicted in step 355. In an embodiment, epidemiological model projection adjustment program 122 outputs the one or more future outcomes as an alert notification. In an embodiment, epidemiological model projection adjustment program 122 outputs the one or more future outcomes to the user through user interface 132 of user computing device 130. In an embodiment, responsive to testing the best performing training model with the selected test data, epidemiological model projection adjustment program 122 outputs the one or more future outcomes.

FIG. 4 is a block diagram illustrating the components of computer device 400, suitable for server 120 and/or user computing device 130 within distributed data processing environment 100 of FIG. 1, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

Computer system 400 includes processor(s) 404, memory 406, cache 416, communications fabric 402, persistent storage 408, input/output (I/O) interface(s) 412, and communications unit 410.

Communications fabric 402 provides communications between memory 406, cache 416, persistent storage 408, input/output (I/O) interface(s) 412, and communications unit 410. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a cross switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Program instructions and data (e.g., software and data 414) used to practice embodiments of the present invention may be stored in persistent storage 408 and in memory 406 for execution and/or access by one or more of the respective computer processor(s) 404 via cache 416. In an embodiment, persistent storage 408 includes a magnetic hard disk drive. Alternatively, or in addition to a magnetic hard disk drive, persistent storage 408 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408. Software and data 414 can be stored in persistent storage 408 for access and/or execution by one or more of the respective processor(s) 404 via cache 416. With respect to user computing device 130, software and data 414 includes user interface 132. With respect to server 120, software and data 414 includes epidemiological model projection adjustment program 122.

Communications unit 410, in these examples, provides for communications with other data processing systems or devices. In these examples, communications unit 410 includes one or more network interface cards. Communications unit 410 may provide communications through the use of either or both physical and wireless communications links. Program instructions and data (e.g., software and data 414) used to practice embodiments of the present invention may be downloaded to persistent storage 408 through communications unit 410.

I/O interface(s) 412 allows for input and output of data with other devices that may be connected to server 120 and/or user computing device 130. For example, I/O interface(s) 412 may provide a connection to external device(s) 418, such as a keyboard, a keypad, a touch screen, and/or some other suitable input device. External device(s) 418 can also include portable computer readable storage media, such as, for example, thumb drives, portable optical or magnetic disks, and memory cards. Program instructions and data (e.g., software and data 410) used to practice embodiments of the present invention can be stored on such portable computer readable storage media and can be loaded onto persistent storage 408 via I/O interface(s) 412. I/O interface(s) 412 also connect to display 420.

Display 420 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

While particular embodiments of the present invention have been shown and described here, it will be understood to those skilled in the art that, based upon the teachings herein, changes and modifications may be made without departing from the embodiments and its broader aspects. Therefore, the appended claims are to encompass within their scope all such changes and modifications as are within the true spirit and scope of the embodiments. Furthermore, it is to be understood that the embodiments are solely defined by the appended claims. It will be understood by those with skill in the art that if a specific number of an introduced claim element is intended, such intent will be explicitly recited in the claim, and in the absence of such recitation no such limitation is present. For a non-limiting example, as an aid to understand, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to embodiments containing only one such element, even when the same claim includes the introductory phrases “at least one” or “one or more” and indefinite articles such as “a” or “an”, the same holds true for the use in the claims of definite articles.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart illustrations and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart illustrations and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart illustrations and/or block diagram block or blocks.

The flowchart illustrations and block diagrams in the Figures illustrate the architecture, functionality, and operation of possible implementations of systems, methods, and computer program products according to various embodiments of the present invention. In this regard, each block in the flowchart illustrations or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each flowchart illustration and/or block of the block diagrams, and combinations of flowchart illustration and/or blocks in the block diagrams, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
gathering, by one or more processors, statistical data related to a disease from one or more regional sources;
clustering, by one or more processors, the statistical data related to the disease according to a plurality of localized regional source similarity criteria and a plurality of region criteria;
building, by one or more processors, a plurality of training models based on the clustered statistical data;
training, by one or more processors, the plurality of training models with a time variant feature;
building, by one or more processors, a plurality of feature vectors based on the plurality of localized regional source similarity criteria and the plurality of region criteria;
training, by one or more processors, the plurality of training models separately against the plurality of feature vectors in order to predict one or more outcomes for each of the plurality of localized regional source similarity criteria and the plurality of region criteria;
selecting, by one or more processors, a best performing training model for each of the plurality of localized regional source similarity criteria and the plurality of region criteria based on a performance criterion;
selecting, by one or more processors, test data for each of the plurality of localized regional source similarity criteria and the plurality of region criteria based on the best performing training model from historical data reported on a desired outcome;
updating, by one or more processors, the best performing training model; and
testing, by one or more processors, the best performing training model with the test data selected to predict the one or more outcomes.

2. The computer-implemented method of claim 1, wherein the statistical data includes at least one of a number of confirmed cases of the disease, a number of confirmed fatalities from the disease, one or more test results, a set of data regarding one or more symptoms of the disease, government policy responses to the disease and social interventions, and a previous history of disease in one or more regions of a country.

3. The computer-implemented method of claim 1, wherein the localized regional source similarity criteria includes phase matching based on a daily case time series curve; similarities in socio-demographic features; comorbidities; mobility; and government policy responses to the disease and social interventions.

4. The computer-implemented method of claim 1, wherein the region criteria by which the statistical data is clustered includes state boundaries and national boundaries.

5. The computer-implemented method of claim 1, wherein building the plurality of feature vectors based on the plurality of localized regional source similarity criteria and the plurality of region criteria further comprises:
inputting, by one or more processors, one or more Susceptible-Exposed-Infectious-Removed projections, one or more autoregressive features, one or more sociodemographic features, and one or more time-dependent features into the plurality of feature vectors.

6. The computer-implemented method of claim 1, wherein the one or more outcomes is one or more of a daily number of confirmed cases of the disease, a daily number of hospitalizations because of the disease, a daily number of intensive care unit beds occupied by one or more patients with the disease, and a daily number of deaths as a result of the disease.

7. The computer-implemented method of claim 1, wherein training the plurality of training models separately against the plurality of feature vectors in order to predict the one or more outcomes for each of the localized regional sources further comprises:
conducting, by one or more processors, a walk-forward expanding window method of a train test split.

8. The computer-implemented method of claim 7, wherein conducting the walk-forward expanding window method of the train test split further comprises:
using, by one or more processors, a grid search in order to find a best scoring model;
using, by one or more processors, mean absolute error as a training scorer; and
predicting, by one or more processors, a final score using R².

9. The computer-implemented method of claim 1, wherein the performance criterion is either a mean absolute percentage error or a root mean square.

10. The computer-implemented method of claim 1, subsequent to testing the best performing training model with the test data selected to predict the one or more future outcomes:
outputting, by one or more processors, the one or more outcomes to a user as an alert notification.

11. A computer program product comprising:
one or more computer readable storage media and program instructions stored on the one or more computer readable storage media, the program instructions comprising:
program instructions to gather statistical data related to a disease from one or more regional sources;
program instructions to cluster the statistical data related to the disease according to a plurality of localized regional source similarity criteria and a plurality of region criteria;
program instructions to build a plurality of training models based on the clustered statistical data;
program instructions to train the plurality of training models with a time variant feature;
program instructions to build a plurality of feature vectors based on the plurality of localized regional source similarity criteria and the plurality of region criteria;
program instructions to train the plurality of training models separately against the plurality of feature vectors in order to predict one or more outcomes for each of the plurality of localized regional source similarity criteria and the plurality of region criteria;
program instructions to select a best performing training model for each of the plurality of localized regional source similarity criteria and the plurality of region criteria based on a performance criterion;
program instructions to select test data for each of the plurality of localized regional source similarity criteria and the plurality of region criteria based on the best performing training model from historical data reported on a desired outcome;
program instructions to update the best performing training model; and
program instructions to test the best performing training model with the test data selected to predict the one or more outcomes.

12. The computer program product of claim 11, wherein building the plurality of feature vectors based on the plurality of localized regional source similarity criteria and the plurality of region criteria further comprises:
program instructions to input one or more Susceptible-Exposed-Infectious-Removed projections, one or more autoregressive features, one or more sociodemographic features, and one or more time-dependent features into the plurality of feature vectors.

13. The computer program product of claim 11, wherein training the plurality of training models separately against the plurality of feature vectors in order to predict the one or more outcomes for each of the localized regional sources further comprises:
program instructions to conduct a walk-forward expanding window method of a train test split.

14. The computer program product of claim 13, wherein conducting the walk-forward expanding window method of the train test split further comprises:
program instructions to use a grid search in order to find a best scoring model;
program instructions to use mean absolute error as a training scorer; and
program instructions to predict a final score using R².

15. The computer program product of claim 11, subsequent to testing the best performing training model with the test data selected to predict the one or more outcomes:
program instructions to output the one or more outcomes to a user as an alert notification.

16. A computer system comprising:
one or more computer processors;
one or more computer readable storage media;
program instructions collectively stored on the one or more computer readable storage media for execution by at least one of the one or more computer processors, the stored program instructions comprising:
program instructions to gather statistical data related to a disease from one or more regional sources;
program instructions to cluster the statistical data related to the disease according to a plurality of localized regional source similarity criteria and a plurality of region criteria;
program instructions to build a plurality of training models based on the clustered statistical data;
program instructions to train the plurality of training models with a time variant feature;
program instructions to build a plurality of feature vectors based on the plurality of localized regional source similarity criteria and the plurality of region criteria;
program instructions to train the plurality of training models separately against the plurality of feature vectors in order to predict one or more outcomes for each of the plurality of localized regional source similarity criteria and the plurality of region criteria;
program instructions to select a best performing training model for each of the plurality of localized regional source similarity criteria and the plurality of region criteria based on a performance criterion;
program instructions to select test data for each of the plurality of localized regional source similarity criteria and the plurality of region criteria based on the best performing training model from historical data reported on a desired outcome;
program instructions to update the best performing training model; and
program instructions to test the best performing training model with the test data selected to predict the one or more outcomes.

17. The computer system of claim 16, wherein building the plurality of feature vectors based on the plurality of localized regional source similarity criteria and the plurality of region criteria further comprises:
program instructions to input one or more Susceptible-Exposed-Infectious-Removed projections, one or more autoregressive features, one or more sociodemographic features, and one or more time-dependent features into the plurality of feature vectors.

18. The computer system of claim 16, wherein training the plurality of training models separately against the plurality of feature vectors in order to predict the one or more outcomes for each of the localized regional sources further comprises:
program instructions to conduct a walk-forward expanding window method of a train test split.

19. The computer system of claim 18, wherein conducting the walk-forward expanding window method of the train test split further comprises:
program instructions to use a grid search in order to find a best scoring model;
program instructions to use mean absolute error as a training scorer; and
program instructions to predict a final score using R².

20. The computer system of claim 16, subsequent to testing the best performing training model with the test data selected to predict the one or more outcomes:
program instructions to output the one or more future outcomes to a user as an alert notification.
